(19) United States
(12) Patent Application Publication
(10) Pub. No.: US 2022/0111395 A1
(43) Pub. Date: Apr. 14, 2022

(54) METHOD FOR RECYCLING ALUMINUM ALLOYS USING CONTAMINANT CONCENTRATION ESTIMATES FOR QUALITY CONTROL

(71) Applicant: House of Metals Company Limited, Toronto (CA)

(72) Inventor: Daniel Bitton, Toronto (CA)

(21) Appl. No.: 17/386,178

(22) Filed: Jul. 27, 2021

Related U.S. Application Data
(60) Provisional application No. 63/090,925, filed on Oct. 13, 2020.

(51) Int. Cl. B02C 19/00; C22B 1/00; B02C 23/08; B02C 23/20; B02C 25/00

(52) U.S. Cl. CPC B02C 19/0062; C22B 1/005; B02C 25/00; B02C 23/20; B02C 23/08

(57) ABSTRACT

A method of recycling aluminum alloy wheels, the method comprising (a) providing a feed of aluminum alloy wheels; (b) fragmenting the aluminum alloy wheels into a plurality of fragments; (c) cleaning the plurality of fragments to at least partly remove at least one contaminant element therefrom; (d) determining a contaminant concentration estimate for each contaminant element in the plurality of fragments; and (e) operating a data processor to either approve or reject the plurality of fragments, based on an aggregate contaminant concentration calculation. When the plurality of fragments is approved, they may be provided to a downstream recycling process. When the plurality of fragments is rejected, they may not be provided to the downstream recycling process without further cleaning.

### CROSS-REFERENCE TO RELATED APPLICATION

This application claims the benefit of Provisional Application Serial No. 63/090,925, filed Oct. 13, 2020, the entirety of which is hereby incorporated by reference.

### FIELD

The described embodiments relate to the field of recycling, and, in particular, to the use of contaminant concentration estimates during the recycling process for quality control.

### BACKGROUND

Recycling what would otherwise be waste materials to form new materials or objects is important in modern waste management. Many different materials can be recycled, for example, glass, paper, cardboard, metal, plastic, tires, textiles, batteries, and electronics. The typical method for recycling waste material includes pickup, sorting, cleaning, and processing.

Metals are of particular value for recycling. Unlike other materials, metals may be recycled into products of substantially similar quality to their feed material.

Slight differences in elemental composition can result in vastly different material properties. Certain high value alloys have specific elemental compositions. Metals provided for recycling may have discrepancies in elemental composition from high value alloys. These discrepancies may be due to contaminants, i.e., debris, deposited on the metals provided for recycling.

### SUMMARY

This summary is intended to introduce the reader to various aspects of the applicant's teaching, but not to define any specific embodiments. In general, disclosed herein are one or more methods of recycling waste metal.

In a first aspect, some embodiments of the invention provide a method of recycling aluminum alloy wheels. The method comprises: (a) providing a feed of aluminum alloy wheels of a particular alloy; (b) fragmenting the aluminum alloy wheels into a plurality of fragments; (c) cleaning the plurality of fragments to at least partly remove at least one contaminant element; (d) for each fragment of a representative sample of fragments of the plurality of fragments, determining, for each contaminant element of the at least one contaminant element, a contaminant concentration estimate for that fragment; (e) operating a data processor to either approve or reject the plurality of fragments, based on an aggregate contaminant concentration calculation for the plurality of fragments, the aggregate contaminant concentration calculation being based on, for each contaminant element of the at least one contaminant element, and for each fragment of the representative sample of fragments, the contaminant concentration estimate for that contaminant element in that fragment. When the plurality of fragments is approved, providing the plurality of fragments to a downstream recycling process to produce a target aluminum alloy; and when the plurality of fragments is rejected, not providing the plurality of fragments to the downstream recycling process to produce the target aluminum alloy without further cleaning to further remove any contaminant in the at least one contaminant element.

According to some aspects of some embodiments of the present invention, the at least one contaminant element is at least two contaminant elements and comprises at least a first contaminant element and a second contaminant element; and for each fragment of the representative sample of fragments, determining, for each contaminant element of the at least two contaminant elements, the contaminant concentration estimate for that fragment, comprises determining a first contaminant concentration estimate for the first contaminant in that fragment and a second contaminant concentration estimate for the second contaminant element in that fragment.

According to some aspects of some embodiments of the present invention, the first contaminant element and the second contaminant element are selected from the group consisting of iron, nickel, chromium, silicon, lead, copper, and zinc.

According to some aspects of some embodiments of the present invention, the method further comprises: selecting the target aluminum alloy, wherein the target aluminum alloy is selected to be of a target alloy composition; the target alloy composition specifies, for each contaminant element of the at least two contaminant elements, a maximum concentration for that contaminant element in the target aluminum alloy, such that the target alloy composition specifies a first maximum concentration for the first contaminant element, and a second maximum concentration for the second contaminant element; the aggregate contaminant concentration calculation for the plurality of fragments, comprises at least two aggregate concentration estimates for the plurality of fragments, the at least two aggregate concentration estimates comprising, for each contaminant element in the at least two contaminant elements, an aggregate concentration estimate for that element in the plurality of fragments. The method may further comprise, for each contaminant element in the at least two contaminant elements, defining a maximum threshold based at least partly on the maximum concentration for that contaminant element in the target aluminum alloy; and determining, for each contaminant element in the at least two contaminant elements, when the maximum threshold for that contaminant element is exceeded by the aggregate contaminant concentration estimate for that contaminant element, such that i) the data processor approves the plurality of fragments when, for each contaminant element in the at least two contaminant elements, the maximum threshold for that contaminant element is not exceeded, and, ii) the data processor rejects the plurality of fragments when the concentration estimate for any contaminant element of the at least two contaminant elements exceeds the maximum threshold for that contaminant element.

According to some aspects of some embodiments of the present invention, providing the plurality of fragments to the downstream recycling process further comprises providing the plurality of fragments with an indication of the target aluminum alloy for use in manufacturing the at least one component made from the target aluminum alloy.

According to some aspects of some embodiments of the present invention, providing the plurality of fragments to the downstream recycling process further comprises providing the plurality of fragments with i) an indication of the target aluminum alloy for use in manufacturing the at least one component made from the target aluminum alloy, and ii) an indication of the at least two aggregate contaminant concentration estimates for the plurality of fragments.

According to some aspects of some embodiments of the present invention, the at least one contaminant element comprises iron; for each fragment of the representative sample of fragments, determining, for each contaminant element of the at least one contaminant element, the contaminant concentration estimate for that fragment, comprises determining an iron concentration estimate; and, the aggregate contaminant concentration calculation for the plurality of fragments is based on, for each fragment of the representative sample of fragments, the iron concentration estimate for that fragment.

According to some aspects of some embodiments of the present invention, when and after the data processor rejects the plurality of fragments based on the aggregate contaminant concentration calculation, the method further comprises operating at least one magnet to separate iron-containing fragments from the plurality of fragments, and then determining, for each fragment of a second representative sample of fragments of the plurality of fragments, the contaminant concentration estimate for that fragment, and then again operating the data processor to either approve or reject the plurality of fragments, based on an aggregate contaminant concentration calculation for the plurality of fragments, the aggregate contaminant concentration calculation being determined from, for each fragment of the second representative sample of fragments, the contaminant concentration estimate for that fragment.

According to some aspects of some embodiments of the present invention, the at least one contaminant element comprises at least one attachment contaminant element, the at least one attachment contaminant element being concentrated in attachments attached to the aluminum alloy wheels in the feed of aluminum alloy wheels; for each fragment of the representative sample of fragments, determining, for each contaminant element of the at least one contaminant element, the contaminant concentration estimate for that fragment, comprises determining at least one attachment contaminant concentration estimate; and, the aggregate contaminant concentration calculation for the plurality of fragments comprises an aggregate attachment contaminant concentration calculation based on, for each fragment of the representative sample of fragments, the attachment contaminant concentration estimate for that fragment.

According to some aspects of some embodiments of the present invention, the at least one attachment contaminant element comprises at least one of iron and lead.

According to some aspects of some embodiments of the present invention, when and after the data processor rejects the plurality of fragments based on the aggregate contaminant concentration calculation, the method further comprises visually inspecting the plurality of fragments to identify at least one additional attachment contaminant element attached to the plurality of fragments, and then removing the at least one additional attachment contaminant element, and then determining, for each fragment of a second representative sample of fragments, the attachment contaminant concentration estimate for that fragment, and then operating the data processor to either approve or reject the plurality of fragments based on a second aggregate contaminant concentration calculation for the plurality of fragments, the second aggregate contaminant concentration calculation being determined from, for each fragment of the second representative sample of fragments, the attachment contaminant concentration estimate for that fragment.

According to some aspects of some embodiments of the present invention, the at least one contaminant element comprises at least one coating contaminant element, wherein the at least one coating contaminant element comprises at least one of nickel, chromium, copper, and zinc; for each fragment of the representative sample of fragments, determining, for each contaminant element of the at least one contaminant element, the contaminant concentration estimate for that fragment, comprises determining, for each coating contaminant element of the at least one coating contaminant element, a coating contaminant concentration estimate for that coating contaminant element; and, the aggregate contaminant concentration calculation comprises an aggregate coating contaminant concentration calculation based on, for each fragment of the representative sample of fragments, and for each coating contaminant element of the at least one coating contaminant element, the coating contaminant concentration estimate for that coating contaminant element.

According to some aspects of some embodiments of the present invention, when and after the data processor rejects the plurality of fragments based on the aggregate contaminant concentration calculation, the method further comprises determining an alternative target alloy based at least partly on the aggregate coating contaminant concentration calculation; and then providing the plurality of fragments to an alternative downstream recycling process to produce the alternative target aluminum alloy.

According to some aspects of some embodiments of the present invention, when and after the data processor rejects the plurality of fragments based on the aggregate contaminant concentration calculation, the method further comprises, determining an alternative target alloy based at least partly on the aggregate contaminant concentration calculation; and then providing the plurality of fragments to an alternative downstream recycling process to produce the alternative target aluminum alloy.

According to some aspects of some embodiments of the present invention, for each fragment of the representative sample of fragments of the plurality of fragments, determining, for each contaminant element of the at least one contaminant element, a contaminant concentration estimate for that fragment comprises heating a material of the fragment to a point where the material will emit a characteristic radiation while cooling down, operating a sensor to detect that characteristic radiation, and operating a processor to analyze the characteristic radiation to determine the composition measurements of the material.

According to some aspects of some embodiments of the present invention, the aggregate contaminant concentration calculation for the plurality of fragments, comprises at least two concentration variance estimates for the plurality of fragments, the at least two concentration variance estimates comprising, for each contaminant element in the at least two contaminant elements, a concentration variance estimate for that contaminant element in the plurality of fragments; and, for each contaminant element in the at least two contaminant elements, the maximum threshold for that contaminant element is determined at least partly based on the concentration variance estimate for that contaminant element in the plurality of fragments.

### BRIEF DESCRIPTION OF THE DRAWINGS

These and other advantages of the instant invention will be more fully and completely understood in conjunction with the following detailed description of embodiments and aspects of the present invention with reference to the following drawings, in which:

FIG. 1, in a flow chart, illustrates a method of recycling waste metal pieces.

FIG. 2, in a flow chart, illustrates a method of recycling aluminum alloy wheels.

### DESCRIPTION OF EXEMPLARY EMBODIMENTS

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

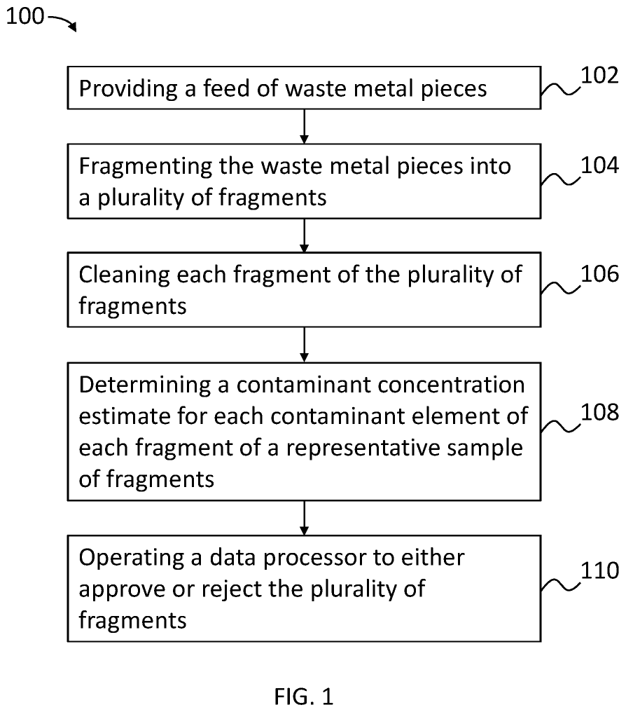

Reference is first made to FIG. 1, in which a method 100 for recycling waste metal pieces is shown. Method 100 begins with providing a feed of waste metal pieces at step 102. The waste metal pieces provided at step 102 are of a particular alloy type. For example, the feed of waste metal pieces may be a feed of waste metal pieces composed of aluminum alloys. In other examples, the feed of waste metal pieces may be a feed of waste metal pieces composed of any one of bismuth alloys, brass alloys, cobalt alloys, copper alloys, gallium alloys, gold alloys, indium alloys, iron alloys, lead alloys, magnesium alloys, mercury alloys, nickel alloys, potassium alloys, silver alloys, steel alloys, tin alloys, titanium alloys, zinc alloys, zirconium alloys, etc.

In some examples of method 100, although each piece of waste metal in the feed of waste metal pieces may be made of the same alloy type, the composition of one piece may differ from the composition of at least one of the other pieces in the feed. In some examples, each piece may be one composition of two different compositions present in the feed. In other examples, each piece of waste metal may be one composition of any number of different compositions present in the feed of waste metal pieces. The composition of one piece may differ from the composition of another piece because alloys of the same type can have any concentration of specific elements within a range. For example, Eccomelt® 356.2 has the following elemental composition requirements: Si: 6.5%-7.5%, Cu: 0%-0.02%, Fe: 0%-0.14%, Mg: 0.25%-0.4%, Zn: 0%-0.018%, Mn: 0%-0.03%, Ni: 0%-0.008%, Cr: 0%-0.03%, Sn: 0%-0.01% Ti: 0%-0.15% Sr: 0%-0.02% Al: 91.674% minimum. Accordingly, even if each piece of waste metal in the entire feed of waste metal pieces were Eccomelt® 356.2, the composition of a first piece in that feed might be different than that of a second piece. Further, the composition of one piece may differ from the composition of another piece due to contaminants that may be located on an external surface of at least one of the pieces.

Accordingly, the feed of waste metal pieces will have an aggregate or batch composition which is based on the different compositions of the different pieces of waste metal, as well as the relative masses of those pieces of waste metal. That is, if all of the pieces of waste metal in a feed of waste metal were to be melted down and mixed to provide a homogeneous mixture, then the composition of that homogeneous mixture would be the batch composition of that feed of waste metal pieces. This batch composition of the feed of waste metal pieces may be unknown when the pieces of waste metal are initially provided.

The pieces of waste metal in this feed of waste metal may all originate from the same kind of components being recycled. For example, a feed of aluminum alloy wheels of a particular alloy, such as aluminum alloy A356.2. Again, despite all the waste metal pieces being of a particular alloy type and possibly from the same kind of component being recycled, they may nonetheless differ slightly in composition.

Material properties may vary significantly with slight variations in composition. Alloys with certain specific elemental compositions may exhibit material properties that are much more desirable than alloys with slightly varying elemental compositions. These material properties may include mechanical strength properties, chemical resistance properties, corrosion resistance properties, and other properties. For example, certain specific elemental compositions may result in a measurably greater mechanical yield strength in tension. Therefore, it may be desirable to produce a batch of cleaned fragments from a feed of waste metal pieces, that, when melted down into a homogenous mixture, has a specific elemental composition.

Referring still to FIG. 1, at step 104, the waste metal pieces are fragmented into a plurality of fragments. The size of fragments produced during the fragmenting process 104 may vary depending on the design and configuration of the fragmenting unit, for example, the size, spacing and orientation of shredders or cutters. When fragmented, each fragment of the plurality of fragments may be just small enough to facilitate removal of contaminants that may be on the surface and/or affixed to that fragment. That is, the fragments may be as large as possible given the need to remove the contaminants.

In some examples, fragments may be produced by passing waste metal pieces through a fragmenting unit. A fragmenting unit may be a shredding apparatus. Any suitable shredder known in the art may be used. For example, waste metal pieces may be supplied to a hopper of a conventional shredding apparatus, such as the SSI Series 45H shredder available from SSI Shredding Systems Inc. at 9760 SW Freeman Drive, Wilsonville, Oreg., 97070-9286, USA. This shredding apparatus may include a cutter box housing cutters, which can be mounted on parallel shafts that rotate horizontally in opposite directions. The feed hopper can be located above the cutter box. Due to the force of gravity, the waste metal pieces placed in the feed hopper can then be fed downwardly into the proper location where they can be engaged by the cutters and torn or cut into shreds.

At step 106, each fragment of the plurality of fragments is cleaned by a cleaning unit or station to at least partly remove at least one contaminant element from that fragment. It is to be understood that not all fragments produced in step 104 may have at least one contaminant element on a surface thereof and/or affixed thereto. However, even if a fragment does not have at least one contaminant element affixed thereto, it may still be cleaned as if it did.

As described above, it may be desirable to at least partially clean contaminant elements from the plurality of fragments to at least lower the concentrations of these contaminant elements in the batch composition if not remove these contaminant elements entirely. Including the contaminant elements in the batch may skew the aggregate batch composition such that this batch has contaminant concentrations too high for the batch to be used in making some valuable alloys, since material properties may be sensitive to elemental composition. Put another way, the feed of waste metal pieces has a batch composition that includes the contaminant elements and therefore may have a batch composition that is undesirable; whereas the cleaned plurality of fragments has a batch composition that includes relatively less contaminant elements and therefore may have a batch composition that is desirable. Accordingly, it may be desirable to remove all contaminant elements to leave behind a bare metal surface, free of contaminant elements. That said, as described in detail below, it may be impractical and unnecessary to remove all contaminant elements from the plurality of fragments.

Example contaminant elements include, but are not limited to, coatings, such as paints, metal electroplating, ceramic coatings, or plastic coatings. Similarly, external surfaces of waste metal pieces may be characterized by corrosion or environmental contamination such as rust. Further, contaminants may be nuts, bolts, screws, steel bushings, etc. (i.e., foreign debris) that may be attached to the waste metal pieces.

In some examples, to clean the plurality of fragments, each fragment of the plurality of fragments may be subjected to shot blasting. That is, the cleaning unit may comprise a shot blasting unit. Alternatively or additionally, each fragment may be subjected to, at the cleaning station, manual hand cleaning by a worker, water blasting, sand blasting, laser cleaning, a washing process, and/or wire brush grinding. In some exemplary methods, at least a portion of the plurality of fragments may be subjected to more than one form of cleaning during the cleaning process.

When using shot blasting, for example, during the cleaning process 106, abrasive particles, i.e. a plurality of shot, can be projected at the fragments at high speed. The shot impacting the surfaces of the fragments can dislodge coatings, corrosion, and debris, i.e., contaminant elements, deposited on the surfaces of the fragments, resulting in fragments with surfaces largely free from contamination.

Shot blasting may be conducted in any suitable shot blasting apparatus. For example, the apparatus may be a centrifugal blasting apparatus, such as the model (FB-4/28/E/MR) Flexbel system available from BCP Wheelabrator of 1219 Corporate Drive, Burlington, Ontario, L7L 5V5, Canada, which is suitable for blast cleaning small parts. Abrasives may include steel shot, alumina, silica, and other abrasive materials.

Within step 106, if shot blasting is used, the plurality of shot blasted fragments may be separated from the plurality of shot (depending on the form of cleaning that is used, other similar separation steps may be conducted). It may be desirable to separate the plurality of fragments from the plurality of shot because including the shot in the aggregate batch might skew the aggregate batch composition so that this batch has contaminant concentrations too high for the batch to be used in making some valuable alloys. Although desirable, in some examples, it may be impractical to completely separate the plurality of fragments from the plurality of shot. That is, in some examples, a portion of the plurality of fragments and the plurality of shot might be separated from the remaining plurality of fragments. Further, in some examples, a portion of the plurality of shot may not be separated from the remaining plurality of fragments.

After being cleaned at step 106, at step 108 a contaminant concentration estimate is determined. In the example illustrated, a contaminant concentration estimate is determined for each contaminant element of each fragment in a representative sample of fragments in the plurality of fragments. In other examples, a contaminant concentration estimate may be determined for each contaminant element for each fragment of the plurality of fragments. A contaminant concentration estimate is an estimate of the amount (by weight) of a contaminant element with respect to the weight of the fragment containing that contaminant element (for example on a surface of that fragment and/or affixed to that fragment). It is to be understood that elements not commonly found within the base alloy are not necessarily considered contaminant elements. Further, what is considered as a contaminant element may vary between recycling processes, depending on, for example, desired characteristics for the batch. That is, for example, in one recycling process of aluminum alloys, copper may be considered as a contaminant element, whereas in a second recycling process, copper may not be considered as a contaminant element. Methods for calculating a contaminant concentration estimate are described in detail below (see, Determining the Contaminant Concentration Estimate).

Since a single fragment may include multiple contaminant elements, in some examples a contaminant concentration estimate may be determined for several contaminant elements of that fragment. For example, a fragment may contain two contaminant elements, i.e., a first contaminant element and a second contaminant element. Accordingly, at step 108, a first contaminant concentration estimate of the first contaminant element may be made and a second contaminant concentration estimate of the second contaminant element may be made. Further, it is to be understood that not all fragments will contain the same contaminant elements. For example, some fragments in a plurality of fragments may be contaminated by paint, some may be contaminated by rust, and some may be contaminated by paint and rust.

Further, although desirable, it may be impractical to determine a contaminant concentration estimate of each contaminant element for each fragment of the plurality of fragments. For example, it may be impractical to test each fragment of the plurality of fragments due to the amount of time required to make a contaminant concentration estimate. Accordingly, in some examples, a subset of the plurality of fragments may be used as a representative sample of fragments of the plurality of fragments, and contaminant concentration estimates for each contaminant element may be determined for only the fragments of the representative sample of fragments.

Next, still referring to FIG. 1, at step 110, the plurality of fragments can be either approved or rejected by a data processor. The data processor can approve or reject the plurality of fragments based on an aggregate contaminant concentration calculation. The aggregate contaminant concentration calculation is based on each contaminant concentration estimate determined at step 108.

For example, the aggregate contaminant concentration calculation may be based on the highest contaminant concentration estimates measured in the representative sample of fragments, or the proportion of fragments having contaminant concentration estimates over a specific threshold. This threshold may be specific to a particular contaminant element. For example, the aggregate contaminant concentration calculation may be based on the proportion of fragments having an iron concentration estimate over a specific threshold. Alternatively, many contaminant elements may be assigned their own specific threshold. For example, the aggregate contaminant concentration calculation may be based on the proportion of fragments having an iron concentration estimate over a specific threshold for iron and/or a lead concentration estimate over a specific threshold for lead.

In some embodiments, the aggregate contaminant concentration calculation may include determining, from individual contaminant concentration estimates for individual fragments for a specific contaminant, a standard deviation or variance for concentrations of that contaminant in the representative sample of fragments. If that standard deviation or variance is too high, then the batch may be rejected as the aggregate contaminant concentration estimate for that contaminant may not be determinable with sufficient certainty. Then, the specific thresholds for a specific contaminant may be determined at least partly based on the variance or standard deviation amongst the contaminant concentration estimates for that contaminant in the representative sample of fragments. The higher the standard deviation, the lower the specific threshold for that contaminant should be set, relative to the maximum allowable concentration of that contaminant in the recycled alloy, to reduce the probability that the actual concentration of that contaminant in the recycled alloy will exceed the maximum allowable concentration.

If a sufficient proportion of fragments in the representative sample of fragments have unduly high contaminant concentration estimates, say over the specific thresholds for those contaminant elements, then this may indicate an upstream problem, such as inadequate cleaning of the fragments, or that some waste metal pieces were included in the batch that should not have been. That is, it may be desirable to reject a plurality of fragments if the aggregate contaminant concentration estimate cannot be estimated with a high enough certainty, and the presence of very contaminated fragments can be an indication that the aggregate contaminant concentration estimate cannot be estimated with a high enough certainty.

For example, consider a representative sample of fragments including n fragments, n being a positive integer. For the representative sample of fragments to adequately represent the plurality of fragments, n should be selected to be sufficiently large. Now say that it has been determined from experience (i.e., from empirical data collected from prior recycling process instances), that if a high enough proportion of fragments in the representative sample of fragments have iron concentrations over a threshold percentage, say 5%, then this suggests (increases the probability) that there is some problem with the upstream supply or cleaning of the plurality of fragments. The number of fragments having an unduly high iron concentration, and their unduly high iron concentrations, may or may not be significant enough to raise the expected aggregate iron concentration for the entire plurality of segments above acceptable limits. However, even if the number of fragments having an unduly high iron concentration, and their unduly high iron concentrations, is insufficient to raise the expected aggregate iron concentration within the entire plurality of fragments above the acceptable limit, it may be desirable to reject the plurality of fragments as the number of fragments in the representative sample of fragments having an unduly high iron concentration may reduce the confidence in the accuracy of the aggregate iron concentration estimate that can be determined for the entirety of the plurality of fragments based only on iron concentration estimates for the fragments of the representative sample of fragments.

In other examples, the aggregate contaminant concentration calculation may be based on an average of all the contaminant concentration estimates measured for each fragment of the representative sample of fragments, i.e., be based on an aggregate contaminant concentration estimate. That is, the decision to approve or reject a plurality of fragments may be made by comparing an average of the contaminant concentration estimates measured for each fragment of the representative sample of fragments to a threshold for each contaminant element. Provided the representative sample of fragments is sufficiently large relative to the entire plurality of fragments, the average of all the contaminant concentration estimates measured for each fragment of the representative sample of fragments, is likely to provide a statistically accurate approximation of the aggregate contaminant concentrations for the entire plurality of fragments.

Any statistical method known in the art may be used to determine the minimum size of a smaller sample population required to statistically represent the larger population such that attributes of the larger population can be inferred from the attributes measured for the smaller population. Statistical methods may also be used to provide uncertainty values of aggregate concentration composition calculations.

If the representative sample of fragments only contains one contaminant element, the aggregate contaminant concentration calculation can be based only on the aggregate contaminant concentration estimates for that contaminant element. If the representative sample of fragments contains, for example, three contaminant elements, the aggregate contaminant concentration calculation can be based on the aggregate contaminant concentration estimates for each of the three contaminant elements. That is, the aggregate contaminant concentration calculation for the plurality of fragments can be based on each of the aggregate contaminant concentration estimates of each contaminant of the representative sample of fragments of the plurality of fragments.

For example, consider a representative sample of fragments that includes ten identically sized fragments, three of which have a contaminant concentration estimate of 5% by weight iron and a contaminant concentration estimate of 3% by weight lead, three of which have a contaminant concentration estimate of 3% by weight iron and a contaminant concentration estimate of 1% by weight lead, two of which have a contaminant concentration estimate of 7% by weight copper, and two of which have a contaminant concentration estimate of 1% by weight iron, a contaminant concentration estimate of 3% by weight copper, and a contaminant concentration estimate of 2% by weight silicon.

In this example, the aggregate contaminant concentration calculation may be based on an average of each of the contaminant concentration estimates for each fragment, i.e., the aggregate contaminant concentration estimate which, for this example, is 2.6% by weight iron, 1.2% by weight lead, 2% by weight copper, and 0.4% by weight silicon.

Accordingly, if for example, the threshold is set to 3% by weight iron, 2% by weight lead, 3% by weight copper, and 1% by weight silicon, the data processor, based on the aggregate contaminant concentration calculation based on the aggregate contaminant concentration estimates, will approve the plurality of fragments. Alternatively, if for example, the threshold is set to 3% by weight iron, 1% by weight lead, 3% by weight copper, and 1% by weight silicon, the data processor, based on the aggregate contaminant concentration calculation based on the aggregate contaminant concentration estimates, will reject the plurality of fragments.

Accordingly, the aggregate contaminant concentration calculation can be based on the aggregate contaminant concentration estimate(s) in different ways: for example, i) by determining, for each contaminant, mean values for the contaminant concentration estimates for individual fragments and/or the variance or standard deviation in these contaminant concentration estimates for individual fragments, or ii) by determining that the aggregate contaminant concentration estimate(s) cannot be estimated with a high enough certainty, because of the presence of very contaminated fragments within the representative sample of fragments (and possibly concerns about possible errors in upstream cleaning or other processing steps).

In some examples, the aggregate contaminant concentration calculation can be a series of calculations used to approve or reject the plurality of fragments. For example, the aggregate contaminant concentration calculation may comprise the following calculations: (a) can each of the aggregate contaminant concentration estimates be estimated with a high enough certainty?; if yes (b) is each aggregate contaminant concentration estimate below a threshold for that contaminant element?; if yes to (a) and (b), approve the plurality of fragments; if no to either one of (a) or (b), reject the plurality of fragments.

When the plurality of fragments is approved, the plurality of fragments may be provided to a downstream recycling process to produce a target alloy. The target alloy may be similar to the base alloy of the feed of waste metal pieces.

When the plurality of fragments is rejected, the plurality of fragments may not be provided to the downstream recycling process to produce the target alloy out of the recycled material without further cleaning to further remove contaminants from the plurality of fragments. That is, the concentration of contaminants may be large enough to skew the aggregate batch concentration(s) away from the concentration(s) required to produce a target alloy. Accordingly, the fragments may need to be further cleaned to bring the aggregate batch concentration to within the desired concentration.

Alternatively, the rejected plurality of fragments may be provided to a downstream recycling process if/when that downstream recycling process has a use for a batch of fragments having an aggregate batch concentration of that batch. That is, the batch may be provided to an alternative downstream recycling process that has a use for the plurality of fragments even though the plurality of fragments may not have the initially desired aggregate batch concentration. For example, the plurality of fragments may be used to produce a recycled aluminum alloy permitting wider ranges of contaminants.

In some examples, the data processor approves or rejects the plurality of fragments based on the aggregate contaminant concentration calculation in view of a pre-determined target alloy. That is, it may be known that a specific alloy of a particular composition is desired. The particular composition may specify, for each contaminant element, a maximum concentration for that element in the target alloy. The data processor can compare the aggregate contaminant concentration calculation to the target alloy composition specifications and approve or reject the plurality of fragments accordingly.

In some examples, a maximum threshold based at least partly on the maximum concentration for that contaminant element in the target alloy may be determined. That is, the data processor may compare the aggregate contaminant concentration calculation to the maximum threshold as opposed to the maximum concentrations specified in the target alloy composition. It may be desirable to use a maximum threshold as opposed to the maximum concentration to account for errors in estimating the contaminant concentration estimates.

For example, the target alloy composition for Eccomelt® 356.2 may specify that the maximum concentration for iron is 0.14%. Therefore, in some examples the data processor may use a threshold of 0.14% when approving or rejecting fragments based on iron concentration. Alternatively, the threshold may be set to a maximum threshold lower than the maximum concentration, for example, the maximum threshold for iron may be 0.10%. By setting the maximum threshold lower than the maximum concentration, the system may account for errors that may occur when estimating the contaminant concentration estimate(s) and/or when using a representative sample of fragments to estimate characteristics of the plurality of fragments.

Accordingly, the data processor can either approve or reject the plurality of fragments based on the aggregate contaminant concentration calculation which can be based on the maximum concentrations defined by the specified target alloy concentration and/or the maximum threshold of contaminant elements for the target alloy.

That is, for each of the contaminant elements, the data processor can determine when a maximum concentration and/or maximum threshold for a contaminant element is exceeded by the aggregate contaminant concentration calculation, such that i) the data processor approves the plurality of fragments when, for each of the contaminant elements, the maximum concentrations and/or maximum threshold for the contaminant elements is not exceeded, and, ii) the data processor rejects the plurality of fragments when the concentration estimate for any contaminant elements exceeds the maximum concentrations and/or maximum threshold for the corresponding contaminant element.

### Determining the Contaminant Concentration Estimate

Any method known in the art to measure the concentration of a contaminant with respect to a fragment to which that contaminant is affixed to and/or on a surface of, may be used. In some examples, a laser scanner can be used to measure the concentration of contaminants in a representative sample of fragments. This can involve using a laser to heat the material at a point on the surface of a representative fragment to a temperature at which that material will emit a characteristic radiation while cooling down. A sensor can then be operated to detect that characteristic radiation to provide a spectrum of signal magnitudes at different frequencies. This spectrum of signal magnitudes at different frequencies can then be analyzed by a computer processor to infer the relative concentrations of different elements within the alloy, as described, for example, in U.S. Pat. No. 10,220,418, incorporated herein by reference. If the type of base alloy is known (i.e., which elements are expected to be detected by the sensor), the computer processor can infer which elements are “contaminant elements” and which are “alloy elements”. Accordingly, the concentration of contaminant elements can be determined.

A single concentration measurement may be made on each fragment of the representative sample of fragments. The location of this measurement may affect the contaminant concentration estimate. For example, if a measurement is made directly on a rust spot, the contaminant concentration estimate will be different than if the measurement, on the same fragment, was made adjacent to the rust spot. Accordingly, in some examples, multiple concentration measurements may be made of each fragment of the representative sample of fragments. That said, the concentration measurements are to be understood as estimates. It is to be understood that if enough measurements are made on enough fragments, based on statistical analysis, an accurate estimate of the contaminant concentrations can be made.

In one example, a “Laser-Induced Breakdown Spectroscopy” (“LIBS”) composition analyzer manufactured by Laser Distance Spectrometry can be adapted as the laser scanner and sensor. The LIBS composition analyzer may include a radiation emitter, such as an Nd:YAG laser. The laser may shine at a frequency ranging from 1 to 20 hertz, thereby raising the temperature of the fragments at the point of contact between the fragment and the laser to above 30,000 degrees Celsius and generating plasma. The plasma may quickly cool down, returning the energized ions to a low energy state. While returning to the low energy state, the ions may emit characteristic radiation. The LIBS composition analyzer may contain one or more sensors that detect the characteristic radiation. A processor may then analyze readings obtained from the sensors and determine from them the concentration of the constituents contained in the material undergoing the temperature change. The processor may be disposed within the composition analyzer. Alternatively, the processor may be a remote processor.

Other suitable composition analyzers may include composition analyzers that use laser spectroscopy or other systems that rely on other methods of inducing characteristic radiation to be emitted by a material of each fragment at a surface of that fragment and detecting and analyzing that characteristic radiation to determine a composition of that material. The composition analyzers may detect the characteristic radiation by using any suitable sensor—for example, suitable sensors may include complementary metal-oxide-semiconductor (CMOS), high density, short channel metal-oxide-semiconductor (HMOS), charge-coupled device (CCD), and other types of sensors.

Suitable composition analyzers may use, for example, radiation emitters such as plasma, electron beam, or any other radiation emitters suitable to heat a material of each fragment in at least one spot on a surface of that fragment to a point where the material will emit a sufficient quantity and quality of characteristic radiation while cooling down so as to permit a sensor to detect that characteristic radiation and to allow for a processor to determine a composition of the material from that characteristic radiation. The composition analyzer can be adapted to withstand continuous use, as well as typical conditions that may be present in a particular waste metal recycling operation. Such conditions may include vibrations resulting from the operation of transfer mechanisms, and dust and other particles produced in the recycling process.

Alternatively, other means of detecting composition not involving measuring characteristic radiation may be used.

### Recycling Aluminum Alloy Wheels

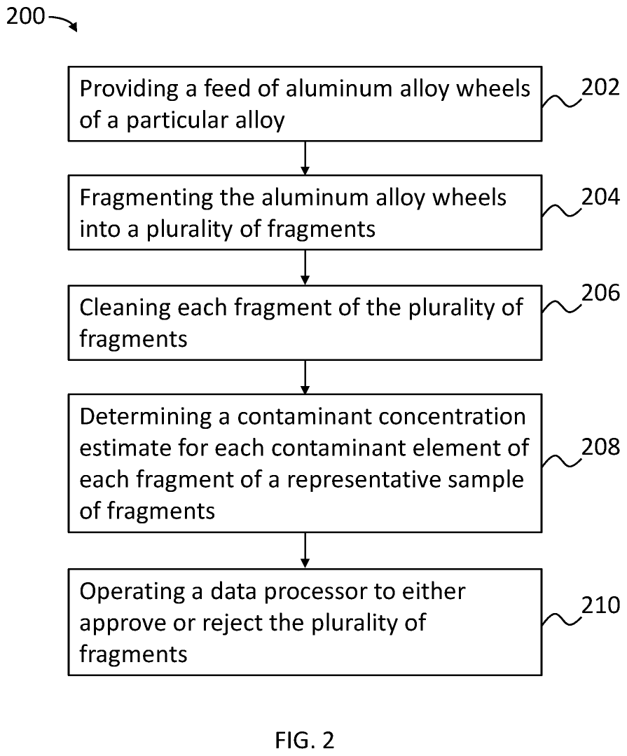

Referring now to FIG. 2, shown therein is method 200 of recycling aluminum alloy wheels. Method 200 of recycling aluminum alloy wheels is an example of an application of method 100 of recycling waste metal pieces. Accordingly, the examples discussed below may be applied to method 100 and the examples discussed above in reference to method 100 can be applied to method 200. Moreover, the discussion below is not meant to limit the methods described herein to that of recycling aluminum alloy wheels. For example, the methods described herein may be applied to a method for recycling objects made of steel alloys, copper alloys, or any other suitable metal.

In step 202 of method 200, a feed of aluminum alloy wheels of a particular alloy is provided. In some examples, this alloy may be A356.2 aluminum alloy. Similar to the waste metal pieces described above, although the aluminum alloy wheels are of a particular alloy, the composition of the wheels may vary. Accordingly, the aggregate composition of a batch of aluminum alloy wheels may be unknown when the batch is initially provided.

In step 204 of method 200, the aluminum alloy wheels may be fragmented into a plurality of fragments. The wheels may be fragmented by running the wheels through a fragmenting unit, such as an industrial shredder. Fragments produced by the fragmenting process may be of substantially uniform size.

In step 206 of method 200, the fragments produced in step 204 are cleaned to at least partially remove at least one contaminant element on a surface thereof and/or affixed thereto. When recycling aluminum alloy wheels, typical contaminant elements include, but are not limited to, iron, nickel, chromium, silicon, lead, copper, and zinc. These are typical elements as they are commonly found in coatings and fasteners that are commonly applied to wheels as well as result from common environmental wear to wheels.

In step 208 of method 200, for each fragment of a representative sample of fragments of the plurality of fragments, and for each contaminant element of at least one contaminant element, a contaminant concentration estimate for that fragment is determined for that contaminant.

In step 210 of method 200, a data processor is operated to either approve or reject the plurality of fragments based on an aggregate contaminant concentration calculation for the plurality of fragments. As described above, when the plurality of fragments is approved, the plurality of fragments may be provided to a downstream recycling process to produce a target aluminum alloy; and when the plurality of fragments is rejected, the plurality of fragments may not be provided to the downstream recycling process to produce the target aluminum alloy without further cleaning to further remove any contaminant in the at least one contaminant element.

In some exemplary methods, the plurality of fragments may be provided to the downstream recycling process with an indication of the target aluminum alloy for use in manufacturing the at least one component made from the target aluminum alloy. The indication of the target aluminum alloy may be determined using the measurements taken to determine the contaminant concentration estimates. That is, the data obtained to determine the contaminant concentration estimates may also be useable to estimate the concentration of the base alloy.

Alternatively, or in addition to the indication of the target aluminum alloy, the plurality of fragments may be provided to the downstream recycling process with an indication of the aggregate contaminate concentration calculation, including, for example the aggregate contaminant concentration estimate(s). It may be desirable for a downstream recycling facility to receive the aggregate contaminant concentration calculation as they may be able to add alloying elements, based on the aggregate contaminant concentration calculation, to the plurality of fragments to alter the composition of the batch.

A system for recycling aluminum alloy rims may comprise series transfer mechanisms in addition to the equipment discussed above, e.g., fragmenting unit, cleaning unit, and composition analyzer. For example, a transfer mechanism may be used to provide the representative sample of fragments to the composition analyzer, for example a laser spectroscopy analyzer. The transfer mechanisms may include one or more of, or a combination of one or more of: a conveyor, a pick-and-place unit, a robotic arm, and other relevant technologies known in the art, selected based on the geometry and size of the rims and/or fragments to be moved. Similar transfer mechanisms may be employed to transport the rims and/or fragments from the composition analyzer to other stations in the recycling process, and between the other stations that may be part of the recycling process.

The data processor may include a computer comprising a non-transient memory and a processor in electronic communication with the non-transient memory. The non-transient memory may have stored thereon a plurality of contaminant thresholds and/or target alloy composition specifications, the computer being in electronic communication with the composition analyzer to receive, for each fragment in the representative sample of fragments of the plurality of fragments, the contaminant composition estimate of that fragment, and the processor being operable to determine the aggregate contaminant concentration calculation and approve or reject the plurality of fragments based thereon.

### Methods for Managing Specific Contaminants

When recycling aluminum alloy wheels, iron may be frequently detected as a contaminant element as shot blasting using iron shot is a common method of cleaning aluminum alloy wheels. As described above, the iron shot may not be separated from the plurality of fragments and, accordingly, can end up in the representative sample of fragments where it may be detected as a contaminant. Having a small amount of iron shot in the representative sample of fragments may cause the data processor to reject the plurality of fragments based on the aggregate contaminant concentration calculation.

If the plurality of fragments is rejected based on the iron concentration of the representative sample of fragments (due to, for example, iron shot), in some example methods, at least one magnet may be operated to remove at least a portion of the iron shot from the fragments (the magnets may also separate out iron containing fragments, generating a remaining plurality of fragments). A second representative sample of fragments may be separated from the remaining plurality of fragments to determine a second aggregate contaminant concentration calculation. The data processor can then approve or reject the remaining plurality of fragments based on the second aggregate contaminant concentration calculation.

High iron levels may also result from contaminates other than shot. And the method of using a magnet to reduce iron concentration in the plurality of fragments (i.e., further cleaning of the fragments) may be used even when all of the shot is separated from the plurality of fragments post cleaning.

When recycling aluminum alloy wheels, contaminant elements that are considered attachment contaminant elements are also frequently detected. Common examples of attachment contaminant elements are nuts, bolts, screws, steel bushings, etc. (i.e., foreign debris) that may be attached to the aluminum alloy wheel when discarded. Attachment contaminant elements may comprise steel, iron, and/or lead and/or other elements.

Attachment contaminant elements are detected when, for example, the LIBS composition analyzer measures a composition that is substantially different than the expected composition of the base alloy. When detecting a contaminant element that is not an attachment element, the measured composition may not be substantially different from the expected composition of the base alloy because contaminates are generally small in comparison to the respective fragment. For example, a fleck of paint is generally small in comparison to a typical fragment, and therefore does not greatly skew the composition measurement away from the expected composition of the base alloy. An attachment contaminant element, on the other hand, may greatly skew the composition measurement away from the expected composition of the base alloy. The data processor when approving or rejecting the plurality of fragments may recognize the presence of an attachment contaminant element in the aggregate contaminant concentration calculation, and may reject the plurality of fragments, accordingly.

When and after the data processor rejects the plurality of fragments based on the aggregate contaminant concentration calculation including an attachment contaminant element, the plurality of fragments may be sent to be visually inspected to locate, identify, and/or remove the attachment contaminant elements attached to at least one of the plurality of fragments.

Once the one or more attachment contaminant elements have been removed from the one or more plurality of fragments, a second representative sample of fragments may be separated from the plurality of fragments. Second contaminate concentration estimate(s) for the plurality of fragments based on the second representative sample of fragments may then be determined. Finally, the data processor may then be operated a second time to either approve or reject the plurality of fragments based on a second aggregate contaminant concentration calculation for the plurality of fragments.

When recycling aluminum alloy wheels, contaminant elements that are considered coating contaminant elements are also frequently detected. Common examples of coating contaminant elements include, but are not limited to, nickel, chromium, copper, and zinc.

Coating contaminant elements can be detected and their composition may be measured by, for example, a LIBS composition analyzer as described above. That is, the at least one contaminant composition estimate for each of the coating contaminant elements can be measured, and these estimates can be used in the aggregate contaminant composition calculation for the plurality of fragments when the data processor approves or rejects the plurality of fragments.

If a plurality of fragments is rejected due to the concentration of coating contaminant elements (for example, a concentration of any single coating contaminant element exceeds a threshold for that element defined by a target alloy composition), it may be counterproductive to send that plurality of fragments for a second cleaning because coating contaminant elements may be very difficult to clean away. Accordingly, rather than cleaning the plurality of fragments a second time in an attempt to bring the plurality of fragments to within the target alloy concentration specification, the method may include determining an alternative target alloy. The alternative target alloy may be selected such that the alternative target alloy concentration specification is consistent with the concentration of coating contaminant elements in the plurality of fragments. If/when an alternative target alloy is determined, the plurality of fragments (with the coating contaminant elements) may be provided to an alternative downstream recycling process to produce products of the alternative target alloy.

The method of selecting an alternative target alloy when the plurality of fragments is outside the target alloy composition specification is not limited to examples when a coating contaminant element brings the plurality of fragments outside the target alloy composition specification. The target alloy composition, and therefore the downstream recycling process, may be changed for any plurality of fragments that is initially rejected by the data processor.

The present invention has been described here by way of example only. Various modification and variations may be made to these exemplary embodiments without departing from the spirit and scope of the invention, which is limited only by the appended claims.

We claim:

1. A method of recycling aluminum alloy wheels, the method comprising:
providing a feed of aluminum alloy wheels of a particular alloy;
fragmenting the aluminum alloy wheels into a plurality of fragments;
cleaning the plurality of fragments to at least partly remove at least one contaminant element;
for each fragment of a representative sample of fragments of the plurality of fragments, determining, for each contaminant element of the at least one contaminant element, a contaminant concentration estimate for that fragment;
operating a data processor to either approve or reject the plurality of fragments, based on an aggregate contaminant concentration calculation for the plurality of fragments, the aggregate contaminant concentration calculation being based on, for each contaminant element of the at least one contaminant element, and for each fragment of the representative sample of fragments, the contaminant concentration estimate for that contaminant element in that fragment;
when the plurality of fragments is approved, providing the plurality of fragments to a downstream recycling process to produce a target aluminum alloy; and
when the plurality of fragments is rejected, not providing the plurality of fragments to the downstream recycling process to produce the target aluminum alloy without further cleaning to further remove any contaminant in the at least one contaminant element.

2. The method as defined in claim 1 wherein
the at least one contaminant element is at least two contaminant elements and comprises at least a first contaminant element and a second contaminant element; and,
for each fragment of the representative sample of fragments, determining, for each contaminant element of the at least two contaminant elements, the contaminant concentration estimate for that fragment, comprises determining a first contaminant concentration estimate for the first contaminant in that fragment and a second contaminant concentration estimate for the second contaminant element in that fragment.

3. The method as defined in claim 2 wherein the first contaminant element and the second contaminant element are selected from the group consisting of iron, nickel, chromium, silicon, lead, copper, and zinc.

4. The method as defined in claim 3 further comprising selecting the target aluminum alloy, wherein
the target aluminum alloy is selected to be of a target alloy composition;
the target alloy composition specifies, for each contaminant element of the at least two contaminant elements, a maximum concentration for that contaminant element in the target aluminum alloy, such that the target alloy composition specifies a first maximum concentration for the first contaminant element, and a second maximum concentration for the second contaminant element;
the aggregate contaminant concentration calculation for the plurality of fragments, comprises at least two aggregate concentration estimates for the plurality of fragments, the at least two aggregate concentration estimates comprising, for each contaminant element in the at least two contaminant elements, an aggregate concentration estimate for that element in the plurality of fragments; and
the method further comprises
for each contaminant element in the at least two contaminant elements, defining a maximum threshold based at least partly on the maximum concentration for that contaminant element in the target aluminum alloy; and
determining, for each contaminant element in the at least two contaminant elements, when the maximum threshold for that contaminant element is exceeded by the aggregate contaminant concentration estimate for that contaminant element, such that i) the data processor approves the plurality of fragments when, for each contaminant element in the at least two contaminant elements, the maximum threshold for that contaminant element is not exceeded, and, ii) the data processor rejects the plurality of fragments when the concentration estimate for any contaminant element of the at least two contaminant elements exceeds the maximum threshold for that contaminant element.

5. The method as defined in claim 1 wherein providing the plurality of fragments to the downstream recycling process further comprises providing the plurality of fragments with an indication of the target aluminum alloy for use in manufacturing the at least one component made from the target aluminum alloy.

6. The method as defined in claim 4 wherein providing the plurality of fragments to the downstream recycling process further comprises providing the plurality of fragments with i) an indication of the target aluminum alloy for use in manufacturing the at least one component made from the target aluminum alloy, and ii) an indication of the at least two aggregate contaminant concentration estimates for the plurality of fragments.

7. The method as defined in claim 1 wherein
the at least one contaminant element comprises iron;
for each fragment of the representative sample of fragments, determining, for each contaminant element of the at least one contaminant element, the contaminant concentration estimate for that fragment, comprises determining an iron concentration estimate; and,
the aggregate contaminant concentration calculation for the plurality of fragments is based on, for each fragment of the representative sample of fragments, the iron concentration estimate for that fragment.

8. The method as defined in claim 7 wherein when and after the data processor rejects the plurality of fragments based on the aggregate contaminant concentration calculation, the method further comprises
operating at least one magnet to separate iron-containing fragments from the plurality of fragments, and then
determining, for each fragment of a second representative sample of fragments of the plurality of fragments, the contaminant concentration estimate for that fragment, and then
again operating the data processor to either approve or reject the plurality of fragments, based on an aggregate contaminant concentration calculation for the plurality of fragments, the aggregate contaminant concentration calculation being determined from, for each fragment of the second representative sample of fragments, the contaminant concentration estimate for that fragment.

9. The method as defined in claim 1 wherein
the at least one contaminant element comprises at least one attachment contaminant element, the at least one attachment contaminant element being concentrated in attachments attached to the aluminum alloy wheels in the feed of aluminum alloy wheels;
for each fragment of the representative sample of fragments, determining, for each contaminant element of the at least one contaminant element, the contaminant concentration estimate for that fragment, comprises determining at least one attachment contaminant concentration estimate; and,
the aggregate contaminant concentration calculation for the plurality of fragments comprises an aggregate attachment contaminant concentration calculation based on, for each fragment of the representative sample of fragments, the attachment contaminant concentration estimate for that fragment.

10. The method as defined in claim 9 wherein the at least one attachment contaminant element comprises at least one of iron and lead.

11. The method as defined in claim 9 or 10 wherein when and after the data processor rejects the plurality of fragments based on the aggregate contaminant concentration calculation, the method further comprises
visually inspecting the plurality of fragments to identify at least one additional attachment contaminant element attached to the plurality of fragments, and then removing the at least one additional attachment contaminant element, and then
determining, for each fragment of a second representative sample of fragments, the attachment contaminant concentration estimate for that fragment, and then
operating the data processor to either approve or reject the plurality of fragments based on a second aggregate contaminant concentration calculation for the plurality of fragments, the second aggregate contaminant concentration calculation being determined from, for each fragment of the second representative sample of fragments, the attachment contaminant concentration estimate for that fragment.

12. The method as defined in claim 1 wherein
the at least one contaminant element comprises at least one coating contaminant element, wherein the at least one coating contaminant element comprises at least one of nickel, chromium, copper, and zinc;
for each fragment of the representative sample of fragments, determining, for each contaminant element of the at least one contaminant element, the contaminant concentration estimate for that fragment, comprises determining, for each coating contaminant element of the at least one coating contaminant element, a coating contaminant concentration estimate for that coating contaminant element; and,
the aggregate contaminant concentration calculation comprises an aggregate coating contaminant concentration calculation based on, for each fragment of the representative sample of fragments, and for each coating contaminant element of the at least one coating contaminant element, the coating contaminant concentration estimate for that coating contaminant element.

13. The method as defined in claim 12 wherein when and after the data processor rejects the plurality of fragments based on the aggregate contaminant concentration calculation, the method further comprises
determining an alternative target alloy based at least partly on the aggregate coating contaminant concentration calculation; and then
providing the plurality of fragments to an alternative downstream recycling process to produce the alternative target aluminum alloy.

14. The method as defined in claim 1 wherein when and after the data processor rejects the plurality of fragments based on the aggregate contaminant concentration calculation, the method further comprises,
determining an alternative target alloy based at least partly on the aggregate contaminant concentration calculation; and then
providing the plurality of fragments to an alternative downstream recycling process to produce the alternative target aluminum alloy.

15. The method as defined in claim 1 wherein, for each fragment of the representative sample of fragments of the plurality of fragments, determining, for each contaminant element of the at least one contaminant element, a contaminant concentration estimate for that fragment comprises heating a material of the fragment to a point where the material will emit a characteristic radiation while cooling down, operating a sensor to detect that characteristic radiation, and operating a processor to analyze the characteristic radiation to determine the composition measurements of the material.

16. The method as defined in claim 4 wherein
the aggregate contaminant concentration calculation for the plurality of fragments, comprises at least two concentration variance estimates for the plurality of fragments, the at least two concentration variance estimates comprising, for each contaminant element in the at least two contaminant elements, a concentration variance estimate for that contaminant element in the plurality of fragments; and,
for each contaminant element in the at least two contaminant elements, the maximum threshold for that contaminant element is determined at least partly based on the concentration variance estimate for that contaminant element in the plurality of fragments.
